(19) United States
(12) Patent Application Publication
(10) Pub. No.: US 2022/0134067 A1
(43) Pub. Date: May 5, 2022

(54) MEDICAL DRAINAGE STENT

(71) Applicant: Micro-Tech (Nanjing) Co., Ltd., Pukou District Nanjing (CN)

(72) Inventors: Derong LENG, Nanjing (CN); Yuqian LI, Nanjing (CN); Yimin SUN, Nanjing (CN); Yidong SUN, Nanjing (CN); Changqing LI, Nanjing (CN); Ning LI, Nanjing (CN); Chunjun LIU, Nanjing (CN)

(73) Assignee: Micro-Tech (Nanjing) Co., Ltd., Pukou District Nanjing (CN)

(21) Appl. No.: 17/576,080

(22) Filed: Jan. 14, 2022

Related U.S. Application Data
(63) Continuation of application No. PCT/CN2019/126083, filed on Dec. 17, 2019.

(30) Foreign Application Priority Data
Jul. 16, 2019 (CN) 201910643952.8

(51) Int. Cl. A61M 27/00; A61F 2/95

(52) U.S. Cl. CPC A61M 27/002; A61M 2205/32; A61F 2002/9528; A61F 2/95

(57) ABSTRACT

A medical drainage stent that includes a stent body, and a proximal portion and a distal portion that are located at two ends of the stent body for connecting a first tissue wall and a second tissue wall in a juxtaposing manner. The stent body is fixed between the first tissue wall and the second tissue wall to provide a drainage channel. The proximal portion of the stent has N extension portions, where a fixed end of the extension portion is fixedly connected with an edge of a proximal end of the stent body, and the other end of the extension portion is a free end in a free state. Structures of the N extension portions at least partially protrude from the stent body in a radial direction, so that the proximal portion of the stent is fixed on the first tissue wall, where N is a positive integer.

### CROSS-REFERENCE TO RELATED APPLICATIONS

This application is a continuation of International Application No. PCT/CN2019/126083, filed on Dec. 17, 2019, which claims priority to Chinese Application No. 201910643952.8, filed on Jul. 16, 2019, both of the above applications are hereby incorporated by reference in their entireties.

### TECHNICAL FIELD

This application relates to the field of medical appliances, and in particular, to a medical drainage stent.

### BACKGROUND

With development of endoscopy and ultrasound endoscopy, inserting a metal stent with help of an endoscopy for the inter-tissue anastomotic drainage technology has been correspondingly developed, such as the pancreatic cyst drainage, the gastrojejunostomy, and the gallbladder stone removal. However, at present, implantation of such stent requires a physician to be skilled and accurate in operations. If the physician is not accurate enough during a release operation, there may be a risk that the stent falls into the abdominal cavity, thus greatly increasing surgery difficulty. Meanwhile, this type of stents is not easy to be taken back to a particular stent recovery cavity after drainage is completed. Therefore, researches on metal stents that are more convenient for surgical operations and inter-tissue drainage becomes an urgent matter, and also has important clinical significance.

At present, while being used for gastrojejunostomy, an existing anastomotic drainage stent in the market, for example, a biliopancreatic stent having an overall hollow structure made of metal wires that is disclosed in the prior art CN204170264U, can effectively enhance connection stability of the stent during use, and can be used for fistula, drainage, and surgical channel establishment between two visceral organs within a cavity. However, regarding this structure, a slight carelessness in releasing the stent may cause the stent to fall into the abdominal cavity. After the stent falls into the abdominal cavity, a flange structure of the existing stent makes the stent inconvenient to be taken back, thus greatly increasing surgery difficulty and risk.

### SUMMARY

This application provides a medical drainage stent, to resolve problems that a slight carelessness in releasing a stent in the prior art may cause the stent to fall into the abdominal cavity and a stent manufacturing process is complicated. A stent is provided, where a proximal end thereof has an extension portion, ensuring regulation and control for a position of a drainage section while releasing the stent. In this way, to a certain extent, risks during a surgery are reduced, a problem that an existing stent is easy to fall into an abdominal cavity while being released may be effectively resolved, and difficulty and risk of a surgery are effectively reduced. At the same time, the extension portion has extraction force, enabling the fixing of the stent. Moreover, the stent is made of metal wires with good biocompatibility (such as nickel-titanium memory alloy wires), and a manufacturing process is simple and feasible.

In addition, this application provides a medical drainage stent. By disposing a flexible portion on a stent body, the stent may be adapted to surgeries of different sizes. In this way, regulation and control for a position of a drainage section while releasing the stent is ensured during a surgery, thus effectively resolving a problem that an existing stent is easy to fall into an abdominal cavity while being released, and effectively reducing operation difficulty.

Embodiments of this application are implemented according to following manners.

A medical drainage stent, including: a stent body, and a proximal portion and a distal portion that are located at two ends of the stent body for connecting a first tissue wall and a second tissue wall in a juxtaposing manner, the stent body being fixed between the first tissue wall and the second tissue wall to provide a drainage channel, wherein the proximal portion of the stent has N extension portions, a fixed end of the extension portion being fixedly connected with an edge of a proximal end of the stent body, and the other end of the extension portion being a free end in a free state; and structures of the N extension portions at least partially protrude the stent body in a radial direction, so that the proximal portion of the stent is fixed on the first tissue wall, wherein N is a positive integer.

By disposing an extension portion that extends from the stent body to the proximal end, a tube body may be long enough in anastomosis tissue at the proximal end when the stent is released, and the stent is not easy to fall into the abdominal cavity, thus ensuring safety when the stent is released. Meanwhile, tissue walls of two organs may support each other and may be connected to each other, preventing the drainage channel from being pulled out.

Preferably, the extension portion is an arc-shaped tube extending spirally along the edge of the proximal end of the stent body; the fixed end of the arc-shaped tube is fixed at the edge of the proximal end of the stent body; the other end is a free end in a free state; and a structure of the arc-shaped tube partially protrudes the stent body in the radial direction, so that the proximal portion of the stent is fixed on the first tissue wall. By disposing an arc-shaped tube extending spirally along the edge of the proximal end of the stent body, while ensuring sufficient extraction force to the tissue wall, the technical effect that the stent does not damage the tissue wall when the stent is supported on the tissue wall is realized.

Preferably, the extension portion extends outward along a radial direction of the stent body, the fixed end thereof is fixed at the edge of the proximal end of the stent body, and the other end is a free end in a free state. Through a structural design with one end fixed and one end in a free state, flexibility of the stent is greatly improved. On one hand, a contact area with the tissue wall is increased, and pull-apart resistance performance is improved; and on the other hand, the extended free end effectively prevents the stent from falling off, and makes the stent easy to be taken back.

Preferably, all the extension portions are evenly arranged at the edge of the proximal end of the stent body, and an included angle between two adjacent extension portions is 360/N degrees, wherein N is a positive integer from 2 to 4. The use of a plurality of extension portions may effectively improve grip strength between the proximal portion and the tissue wall.

Preferably, the free end of the extension portion is provided with a fixing structure to fixedly connect the free end to the first tissue wall, wherein the fixing structure and the free end are fixedly or not fixedly connected, and supporting force of the fixing structure is greater than that of the extension portion.

Preferably, the stent further includes at least one of the following structures:

(1) a retrieval loop provided at the proximal portion and/or the distal portion of the stent; and

(2) a developing member provided on the stent for displaying a position of the stent under rays.

This application further provides a medical drainage stent, including: a stent body, and a proximal portion and a distal portion that are located at two ends of the stent body for connecting a first tissue wall and a second tissue wall in a juxtaposing manner, the stent body being fixed between the first tissue wall and the second tissue wall to provide a drainage channel, wherein the stent body has a flexible portion whose supporting force is smaller than that of the proximal portion and the distal portion; and after being pressed, the flexible portion can form a recess with a reduced inner diameter, so that structures of the proximal portion and the distal portion of the stent partially protrude from the stent body, realizing that the proximal portion of the stent is fixed on the first tissue wall, and the distal portion is fixed on the second tissue wall. A design of directly disposing the flexible portion on the stent body greatly simplifies a structure of the stent. The flexible portion may effectively reduce stimulation to the tissue, and meanwhile has a function of juxtaposing the tissue. At the same time, compared with a mushroom head, an umbrella-shaped flange, and other conventional designs, a flexible recess structure of the stent may be compressed into a finer implanter, and may be clinically adapted to finer endoscopic channel.

Preferably, the stent body has more than two flexible portions that extend along an axial direction of the stent body, and the flexible portions are distributed at equal intervals in a circumferential direction of the stent body.

Preferably, the flexible portion covers a part of a region of the stent body, and supporting force of the remaining region of the stent body is greater than that of the flexible portion. The flexible portion may be a mesh structure made of a polymer elastic membrane, or braided by wires made of a polymer material, or formed by 3D printing or engraving. The overall supporting force of the flexible portion is smaller than that of the other region of the stent body. In this case, when tissue walls of different organs are juxtaposed, only the flexible portion is concave, and the tissue wall is clamped at an edge of the recess. In this way, the stent connects different tissue walls in a juxtaposing manner.

Preferably, the proximal portion and the distal portion of the stent are provided with fixing structures at positions protruding from the stent body. In this way, juxtaposing effects between the stent and the tissue wall are better.

Preferably, the stent further includes at least one of the following structures:

(1) a retrieval loop provided at the proximal portion and/or the distal portion of the stent; and

(2) a developing member provided on the stent for displaying a position of the stent under rays.

### BRIEF DESCRIPTION OF DRAWINGS

To more clearly describe the technical solutions of the embodiments of this application, the accompanying drawings to be used in the embodiments of this application are briefly illustrated below. It should be understood that the following accompanying drawings merely show some embodiments of this application, and therefore should not be considered as a limitation to the scope. A person of ordinary skills in the art can also derive other related accompanying drawings according to these accompanying drawings without an effective effort.

FIG. 1 is a side view of a stent according to embodiment 1 of the application;

FIG. 2 is a top view of a stent according to embodiment 1 of the application;

FIG. 3 is a top view of a stent according to embodiment 2 of the application;

FIG. 4 is a side view of a stent according to embodiment 3 of the application;

FIG. 5 is a side view when a stent connects tissue walls in a juxtaposing manner according to embodiment 3 of the application;

FIG. 6 is a side view of a stent according to embodiment 4 of the application;

FIG. 7 is a schematic diagram of different states when a fixing structure in a stent according to embodiment 4 of the application is implanted into an organ;

FIG. 8 is a side view of a stent according to embodiment 5 of the application;

FIG. 9 is a front view of a flexible portion of a stent according to embodiment 5 of the application;

FIG. 10 is a top view of a stent according to embodiment 5 of the application;

FIG. 11 is a side view when a stent according to embodiment 5 of the application connects tissue walls in a juxtaposing manner;

FIG. 12 is a side view of a stent according to embodiment 6 of the application;

FIG. 13 is a top view of a stent according to embodiment 6 of the application;

FIG. 14 is a side view of a stent according to embodiment 7 of the application;

FIG. 15 is a perspective view of a stent according to embodiment 8 of the application;

FIG. 16 is a radial sectional view of a stent according to embodiment 8 of the application; and

FIG. 17 is a radial sectional view of a stent according to embodiment 9 of the application.

### DETAILED DESCRIPTION OF THE EMBODIMENTS

The technical solutions in the embodiments of this application are described with reference to the accompanying drawings in the embodiments of this application.

I. Explanations of Conventional Concepts in the Present Application

1. In this application, a stent is a self-expandable stent.

2. In this application, the stent may form an anastomosis between any two adjacent organs in the stomach, the small intestine (the duodenum, the jejunum, and the ileum), the large intestine (the cecum and the colon), the rectum, the biliary structure, or the esophagus. The two organs respectively have a first tissue wall and a second tissue wall. The stent may form a passage between the first tissue wall and the second tissue wall corresponding to the two organs, promoting different types of fluids, such as undigested, partially digested, or fully digested food, to flow through a drainage channel within the stent.

3. In this application, the stent may be braided by a wire made of any metal or alloy material suitable for application in the human body, and preferably is braided by nickel-titanium memory alloy wire, having a mesh structure.

4. In this application, a proximal portion of the stent has a retrieval loop 4. The retrieval loop 4 may be configured to adjust a position, of a drainage channel 5 in the stent, relative to a first tissue wall 6 and a second tissue wall 7 of two adjacent organs, and may be used to take back the stent

5. In this application, the entire stent is attached with a membrane structure, and is configured to prevent a drainage substance from leaking from a mesh of the stent, and prevent growth of a granulation, thereby facilitating to take back the stent later.

6. In this application, the distal portion may be any conventional protruding structure that is clamped with a tissue wall, such as an edge-flange structure formed after being turned outward and curled, or a mushroom head structure, or an umbrella-shaped flange structure.

7. In this application, the proximal portion and the distal portion of the stent may be connected to tissue walls of two different organs in a juxtaposing manner, so as to be connected to different organs. The stent body forms a drainage channel. The proximal end in this application refers to an end close to an operator, and the distal end refers to an end far away from the operator.

8. In this application, an axial direction refers to a direction extending along a longitudinal-axis direction of the stent body. A radial direction refers to a direction extending perpendicular to the longitudinal axis of the stent body. An internal portion of the stent refers to an internal space surrounded by the stent body. An external portion of the stent refers to a space that is outside a tube wall of the stent body and is relative to the internal portion of the stent.

II. Specific Implementations of a Stent Structure in this Application

### Embodiment 1

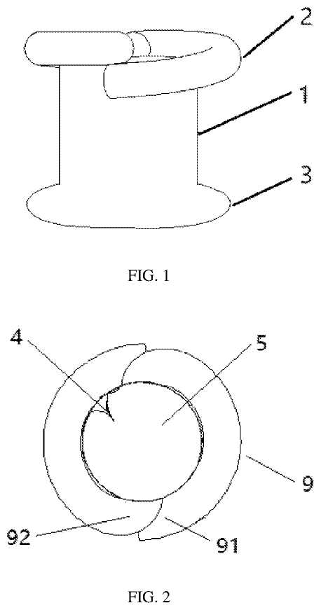

A medical drainage stent shown in FIG. 1 and FIG. 2 includes a stent body 1, and a proximal portion 2 and a distal portion 3 that are located at two ends of the stent body 1 for connecting a first tissue wall and a second tissue wall in a juxtaposing manner. The stent body 1 is fixed between the first tissue wall and the second tissue wall to provide a drainage channel. The proximal portion 2 of the stent has two extension portions 9, wherein a fixed end 92 of the extension portion 9 is fixedly connected with an edge of a proximal end of the stent body 1, and the other end of the extension portion 9 is a free end 91 in a free state. The two extension portions 9 basically form an annular shape surrounding the edge of the proximal end of the stent body 1. Moreover, an overall outer diameter of a circular ring formed by the two extension portions 9 is greater than an outer diameter of the stent body, so that the proximal portion of the stent is able to be fixed on the first tissue wall. In this embodiment, the extension portion 9 is an arc-shaped tube extending spirally along the edge of the proximal end of the stent body 1. The fixed end 92 of the arc-shaped tube is fixed at the edge of the proximal end of the stent body 1, and the other end is the free end 91 in a free state. A structure of the arc-shaped tube partially protrudes from the stent body 1 in the radial direction, so that the proximal portion 2 of the stent is able to be fixed on the first tissue wall. In addition, to obtain better pull-apart force, prevent the stent from falling off during use, and make it easier to take the stent out during pulling off, it is preferable that a partial region (at least occupying ⅓ of a total length of the arc-shaped tube) of the extension portion 9 is fixedly connected with the edge of the proximal end of the stent body 1 along a spiral direction of the extension portion 9 from the fixed end of the extension portion 9, to enhance connection strength between the extension portion and the stent body and improve anti-deformation performance of the stent. In addition, an arc-shaped round tube design of the extension portion reduces stimulation of the stent to the tissue wall, and is less harmful to the human body during a pull-off process. Meanwhile, a tube-shaped design has better supporting effect, can ensure a firm connection between the proximal portion of the stent and the tissue wall, and ensures stability of the stent during a drainage process.

For fixing and clamping the distal end of the stent to the second tissue wall 7, the distal portion 3 of the stent has a curled flange structure. Moreover, it is preferable that an outer diameter of a flange is more than 1.2 times of an outer diameter of the stent body 1, so as to ensure that the distal end of the stent has sufficient pull-apart resistance performance, preventing the stent from being pulled off and falling into an abdominal cavity during use. Meanwhile, the stent in this embodiment is attached with a membrane structure, and the membrane structure may be made of a polymer material with good biocompatibility, such as a PTFE membrane or a silicone membrane. The proximal portion 2 of the stent is provided with a retrieval loop 4. The retrieval loop 4 may be directly connected onto the extension portion 9 of the proximal portion 2 of the stent, or may be connected at an edge of a proximal end of a drainage channel 5 of the stent, provided that the retrieval loop 4 can drag the stent out of the body cavity. The retrieval loop 4 may be made of a polymer material with good biocompatibility, such as a PP thread or a PE thread. To make sure that the retrieval loop can effectively pull off the stent, breaking force of the retrieval loop 4 in this embodiment is preferably greater than 10 N. The stent is provided with a developing member for displaying a position of the stent under rays, wherein a tantalum mark that can be positioned under X-ray is preferable. In this embodiment, the extension portion 9 is a semi-circular arc-shaped ring structure in which two free ends and fixed ends are disposed symmetrically about a center of a cross section of the stent body, or may be replaced with a tubular arc-shaped structure in which three or four free ends and fixed ends are sequentially disposed at equal intervals and interlayer spacing. In this way, effective fixed connection of the tissue wall may also be achieved.

Specific operations of the foregoing stent during use are as follows: delivering the stent to two to-be-juxtaposed organs by a stent delivery device; at beginning of releasing the stent, attaching the distal portion 3 of the stent to a distal side of the second tissue wall 7 of an organ at the distal end; and gradually releasing the stent until the stent is completely released, and ensuring that the proximal portion 2 of the stent fits with a proximal side of the first tissue wall 6 of an organ at the proximal end. In this way, an entire release process of the stent is completed, and the first tissue wall 6 and the second tissue wall 7 form a communication passage by the drainage channel in the stent.

### Embodiment 2

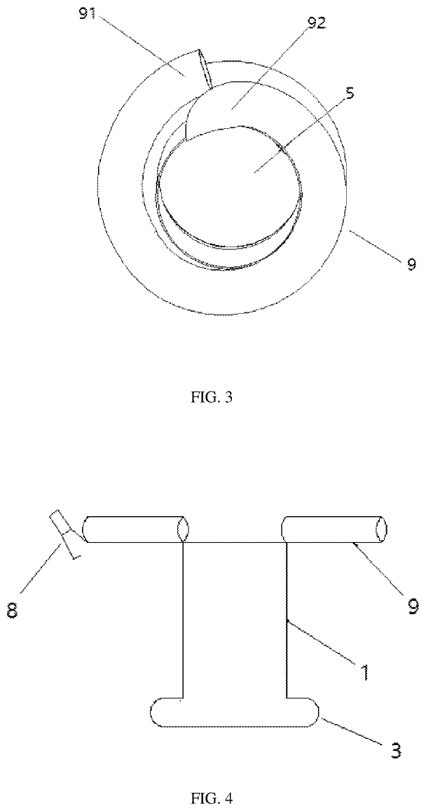

Referring to a medical drainage stent shown in FIG. 3, on the basis of embodiment 1, the two extension portions in embodiment 1 is replaced with one extension portion. To be specific, the medical drainage stent in embodiment 2 includes a stent body 1, and a proximal portion 2 and a distal portion 3 that are located at two ends of the stent body 1 for connecting a first tissue wall and a second tissue wall in a juxtaposing manner. The stent body 1 is fixed between the first tissue wall and the second tissue wall to provide a drainage channel. The proximal portion 2 of the stent has one extension portion 9, wherein a fixed end 92 of the extension portion 9 is fixedly connected with an edge of a proximal end of the stent body 1, and the other end of the extension portion 9 is a free end 91 in a free state. In this embodiment, the extension portion 9 is an arc-shaped tube 1 extending spirally for about 360 degrees along the edge of the proximal end of the stent body 1. The fixed end 92 of the arc-shaped tube is fixed at the edge of the proximal end of the stent body 1, and the other end is the free end 91 in a free state. A structure of the arc-shaped tube partially protrudes from the stent body 1 in the radial direction, so that the proximal portion 2 of the stent is able to be fixed on the first tissue wall. In addition, to obtain better pull-apart force, prevent the stent from falling off during use, and make it easier to take the stent out during pulling off, a partial region (at least occupying ⅓ of a total length of the arc-shaped tube) of the extension portion 9 formed by one arc-shaped tube is fixedly connected with the edge of the proximal end of the stent body 1 along a spiral direction of the extension portion 9 from the fixed end 92 of the extension portion 9, to enhance connection strength between the extension portion and the stent body and improve anti-deformation performance of the stent.

### Embodiment 3

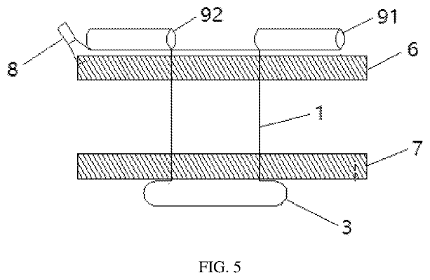

A medical drainage stent shown in FIG. 4 and FIG. 5 includes a stent body 1, an extension portion 9 located at a proximal end of the stent body 1, and a distal portion 3, for connecting a first tissue wall and a second tissue wall in a juxtaposing manner. The stent body 1 is fixed between a first tissue wall 6 and a second tissue wall 7 to provide a drainage channel. The proximal end of the stent has two extension portions 9, wherein a fixed end 92 of the extension portion 9 is fixedly connected with an edge of the proximal end of the stent body 1, and the other end of the extension portion 9 is a free end 91 in a free state. The extension portion extends outward along a radial direction of the stent body 1, wherein the fixed end 92 of the extension portion is fixed at the edge of the proximal end of the stent body 1, and the other end is the free end 91 in a free state. Through a structural design with one end fixed and one end in a free state, flexibility of the stent is greatly improved. On one hand, a contact area with a tissue wall is increased, and pull-apart resistance performance is improved; and on the other hand, the extended free end 91 effectively prevents the stent from falling off, and makes the stent easy to be taken back.

In this embodiment, it is preferable to use a fixing structure 8 to fix and clamp the free end of the extension portion 9 to the first tissue wall, thereby greatly improving connection strength when the stent connects tissue walls of two organs in a juxtaposing manner, and improving surgery stability and reliability. The fixing structure 8 is an independent structure, and is applied to a to-be-connected position by using another surgical instrument. In addition, to improve connection stability between the free end 91 and the tissue wall, supporting force of the fixing structure 8 is preferably greater than that of the extension portion 9. The fixing structure may be an existing fastening structure such as a hemostatic clamp or an anastomosis clamp.

### Embodiment 4

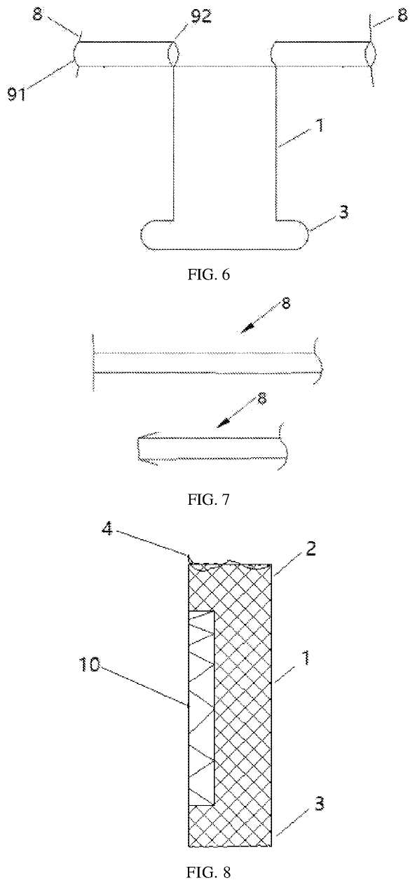

Referring to a medical drainage stent shown in FIG. 6 and FIG. 7, on the basis of embodiment 3, the fixing structure 8 (such as a hemostatic clamp) in embodiment 3 that is not fixedly connected to the extension portion is replaced with a wing-shaped structure that is directly fixedly connected to the extension portion 9. To be specific, a medical drainage stent in this embodiment includes a stent body 1, an extension portion 9 located at a proximal end of the stent body 1, and a distal portion 3, for connecting a first tissue wall and a second tissue wall in a juxtaposing manner. The stent body 1 is fixed between a first tissue wall 6 and a second tissue wall 7 to provide a drainage channel. The proximal end of the stent has two extension portions 9, wherein a fixed end 92 of the extension portion 9 is fixedly connected with an edge of the proximal end of the stent body 1, and the other end of the extension portion 9 is a free end 91 in a free state. The extension portion extends outward along a radial direction of the stent body 1, wherein the fixed end 92 of the extension portion is fixed at the edge of the proximal end of the stent body 1, and the other end is the free end 91 in a free state.

As shown in FIG. 6, a wing-shaped fixing structure 8 extends out from the free end 91. In other words, a wing-shaped structure extends along an inner tube of the tube-shaped extension portion 9 to the free end 91, and extends out from a nozzle of the free end 91 and radially spreads outwards. The wing-shaped fixing structure 8 may be specifically shown in FIG. 7. A main body of the wing-shaped fixing structure 8 is a smooth and flexible hollow or solid tube. The fixing structure 8 has a wing-shaped structure at one end extending out from the free end 91 of the extension portion 9. During use, the end provided with the wing-shaped structure is inserted into a tube-shaped structure of the extension portion 9. At this time, the wing-shaped structure is compressed by a tube wall of the extension portion 10 and yields (as shown in FIG. 7 below). After the wing-shaped structure is pushed to extend out from the free end 91, the wing-shaped structure unfolds based on memory effects of a material thereof. In this way, the wing-shaped structure is clamped with the tissue wall. The fixing structure 8 may be made of nickel-iron memory alloy wires or silicone elastomer.

### Embodiment 5

A medical drainage stent shown in FIG. 8 to FIG. 11 includes a stent body 1, and a proximal portion 2 and a distal portion 3 that are located at two ends of the stent body 1 for connecting a first tissue wall 6 and a second tissue wall 7 in a juxtaposing manner. The stent body 1 is fixed between the first tissue wall 6 and the second tissue wall 7 to provide a drainage channel 5. A proximal end and a distal end of the stent both have retrieval loops 4. During an operation process, the retrieval loop 4 at the distal end may be dragged from internal of the drainage channel 5, so as to constrict an opening of the distal end and pull the retrieval loops 4 out of a human body. This process may effectively reduce friction between the retrieval loop and human tissue, and improve safety of the stent during use. The stent body 1 has an elongated flexible portion 10 that extends along an axial direction of the stent body. Supporting force of the flexible portion 10 is smaller than that of the proximal portion and the distal portion of the stent. After being pressed, the flexible portion 10 may form a recess with a reduced inner diameter, so that structures of the proximal portion 2 and the distal portion 3 of the stent partially protrude from the stent body 1, and that the proximal portion 2 of the stent is fixed on the first tissue wall 6, and the distal portion 3 is fixed on the second tissue wall 7. A design of directly disposing the flexible portion 10 on the stent body 1 greatly simplifies a structure of the stent. The flexible portion 10 may effectively reduce stimulation to the tissue, and meanwhile has a function of juxtaposing the tissue. At the same time, compared with a mushroom head, an umbrella-shaped flange, and other conventional designs, a flexible recess structure of the stent may be compressed into a thinner implanter, and may be clinically adapted to a thinner endoscopic channel. In this embodiment, the flexible portion 10 is preferably braided by wires made of a polymer material, has better flexibility and is more easily deformed than other parts of the stent body 1, and has smaller supporting force. In this case, the flexible portion 10 may radially collapse and concave inward when compressed by tissue, so that the stent connects the two tissue walls in a juxtaposing manner.

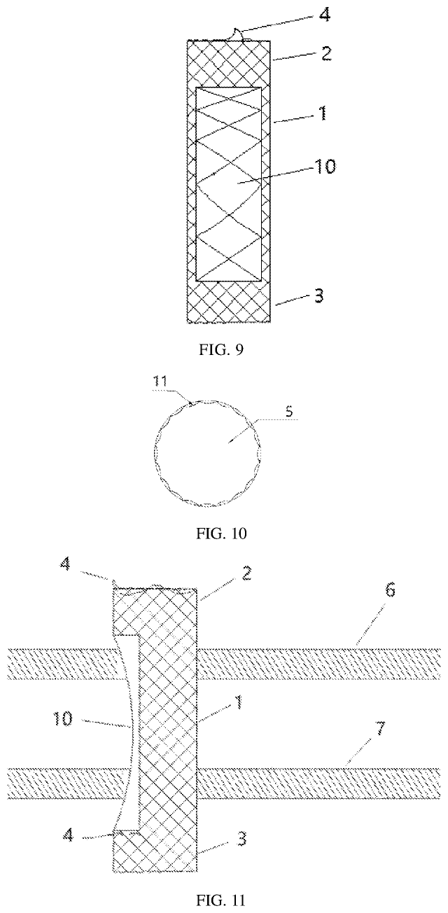

In this embodiment, both end portions of the stent are integrally formed structures that axially extend from the stent body 1 towards the proximal end and the distal end. The stent body 1 is braided by a wire material whose anti-deformation performance is better than that of the flexible portion 10. In addition, as shown in FIG. 10, it may be seen from the top view of the stent that along a circumferential direction, the end portion of the stent has a wire-braided corner 11 that radially shrinks towards a longitudinal axis of the stent. By forming a radially constricted nozzle structure at the two end portions of the stent, it may be implemented that when the stent is released on tissue walls of two adjacent organs, a contact area between the edge of the end portion of the stent and the tissue is reduced, thereby effectively avoiding stimulation to the tissue wall.

### Embodiment 6

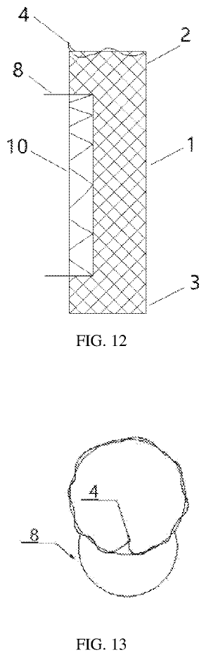

As shown in FIG. 12 and FIG. 13, on the basis of embodiment 5, fixing structures 8 are respectively disposed at a distal end and a proximal end of the flexible portion 10. To be specific, a medical drainage stent in this embodiment includes a stent body 1, and a proximal portion 2 and a distal portion 3 that are located at two ends of the stent body 1 for connecting a first tissue wall 6 and a second tissue wall 7 in a juxtaposing manner. The stent body 1 is fixed between the first tissue wall 6 and the second tissue wall 7 to provide a drainage channel 5. A proximal end of the stent has a retrieval loop 4. The stent body 1 has a flexible portion 10 whose supporting force is smaller than that of the proximal portion and the distal portion. After being pressed, the flexible portion 10 may form a recess with a reduced inner diameter, so that structures of the proximal portion 2 and the distal portion 3 of the stent partially protrude from the stent body 1, and that the proximal portion 2 of the stent is fixed on the first tissue wall 6, and the distal portion 3 is fixed on the second tissue wall 7.

As shown in FIG. 13, in this embodiment, two wing-shaped fixing structures 8 are respectively fixedly connected with the distal end and the proximal end of the flexible portion 10, thereby improving overall supporting force of the stent, to ensure that the stent would not fall in a human body during use. The wing-shaped fixing structure 8 may be made of nickel-titanium wires, and is accommodated in the stent body when the end portion of the stent is in a state of to-be-released. When the end portion of the stent is released, the wing-shaped fixing structure 8 is released and extends from the stent body towards outside of the stent body based on own shape memory properties, and forms an arc-shaped extension structure radially protruding from the stent body in FIG. 13. Supporting force of the wing-shaped fixing structure 8 is greater than that of the flexible portion 10 of the stent, to make sure that both ends of the stent effectively fixedly connect the two tissue walls without being pulled apart.

### Embodiment 7

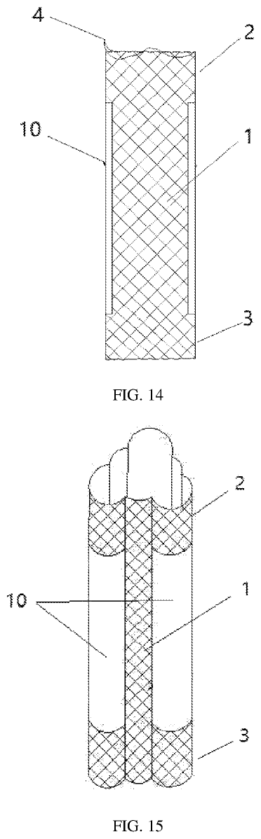

As shown in FIG. 14, on the basis of embodiment 5, two elongated flexible portions 10 that axially extend along an axial direction of the stent body are symmetrically disposed on the stent body along the axial direction, and a polymer wire-braided structure of the flexible portion 10 is replaced with a polymer membrane structure. To be specific, a medical drainage stent in this embodiment includes a stent body 1, and a proximal portion 2 and a distal portion 3 that are located at two ends of the stent body 1 for connecting a first tissue wall 6 and a second tissue wall 7 in a juxtaposing manner. The stent body 1 is fixed between the first tissue wall 6 and the second tissue wall 7 to provide a drainage channel 5. A proximal end of the stent has a retrieval loop 4. The stent body 1 is provided with two elongated flexible portions 10 that are axially symmetrical and extend along the axial direction of the stent body. Supporting force of the flexible portion 10 is smaller than that of the proximal portion and the distal portion. After being pressed, the flexible portion 10 may form a recess with a reduced inner diameter, so that structures of the proximal portion 2 and the distal portion 3 of the stent partially protrude from the stent body 1, and that the proximal portion 2 of the stent is fixed on the first tissue wall 6, and the distal portion 3 is fixed on the second tissue wall 7.

In this embodiment, the flexible portion 10 is preferably a membrane structure made of a polymer material. The polymeric membrane has better flexibility and is more easily deformed than other parts of the stent body 1, and has smaller supporting force. In this case, the flexible portion 10 may radially collapse and concave inward when compressed by tissue, so that the stent connects the two tissue walls in a juxtaposing manner. A material of the polymeric membrane may be any polymer material suitable for the human body, provided that requirements of the foregoing application environments may be satisfied.

### Embodiment 8

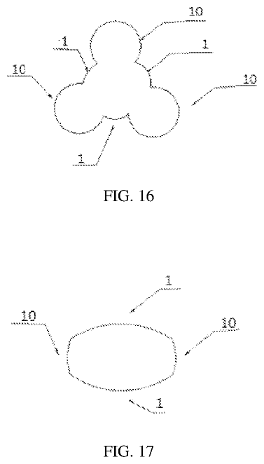

As shown in FIG. 15 and FIG. 16, on the basis of embodiment 7, three elongated flexible portions 10 that axially extend along the axial direction of the stent body are disposed on the stent body at equal intervals. To be specific, a medical drainage stent in this embodiment includes a stent body 1, and a proximal portion 2 and a distal portion 3 that are located at two ends of the stent body 1 for connecting a first tissue wall 6 and a second tissue wall 7 in a juxtaposing manner. The stent body 1 is fixed between the first tissue wall 6 and the second tissue wall 7 to provide a drainage channel 5. A proximal end of the stent has a retrieval loop 4. Three elongated flexible portions 10 that axially extend along the axial direction of the stent body are disposed on the stent body at equal intervals. Supporting force of the flexible portion 10 is smaller than that of the proximal portion and the distal portion. After being pressed, the flexible portion 10 may form a recess with a reduced inner diameter, so that structures of the proximal portion 2 and the distal portion 3 of the stent partially protrude from the stent body 1, and that the proximal portion 2 of the stent is fixed on the first tissue wall 6, and the distal portion 3 is fixed on the second tissue wall 7.

It may be seen from the radial sectional view of the stent shown in FIG. 16 that three elongated flexible portions 10 that axially extend along the axial direction of the stent body are disposed on the stent body at equal intervals. The three flexible portions 10 are distributed on an outer circumference of the stent body 1 at equal intervals. An arc-shaped curvature of a structure of the flexible portion is greater than that of the stent body, thus forming an arc-shaped cylindrical structure that protrudes from an outer wall of the stent body. Through this odd-form cylindrical structure, a contact area between the flexible portion 10 and the tissue wall may be increased, thereby increasing an area of the fixing structure through which both ends of the stent are clamped with the tissue wall, and improving pull-apart resistance performance of the stent. Certainly, due to different actual treatment positions, the flexible portions may be increased or decreased as required, and a quantity of arc-shaped cylindrical structures having the flexible portion 10 is preferably from 2 to 6.

### Embodiment 9

As shown in FIG. 17, on the basis of embodiment 7, a radial section of the stent body is in a nearly rectangular shape, and the flexible portions are symmetrically disposed at short sides. To be specific, a medical drainage stent in this embodiment includes a stent body 1, and a proximal portion 2 and a distal portion 3 that are located at two ends of the stent body 1 for connecting a first tissue wall 6 and a second tissue wall 7 in a juxtaposing manner. The stent body 1 is fixed between the first tissue wall 6 and the second tissue wall 7 to provide a drainage channel 5. A proximal end of the stent has a retrieval loop 4. The stent body 1 is provided with two elongated flexible portions 10 that are axially symmetrical and extend along an axial direction of the stent body. Supporting force of the flexible portion 10 is smaller than that of the proximal portion and the distal portion. After being pressed, the flexible portion 10 may form a recess with a reduced inner diameter, so that structures of the proximal portion 2 and the distal portion 3 of the stent partially protrude from the stent body 1, and that the proximal portion 2 of the stent is fixed on the first tissue wall 6, and the distal portion 3 is fixed on the second tissue wall 7.

It may be seen from the radial sectional view of the stent shown in FIG. 17 that the flexible portions 10 are axially and symmetrically disposed at two sides of the stent body 1, the radial section is in a nearly rectangular shape, and the flexible portions 10 are disposed at short sides. A long side of the rectangle is arc-shaped to reduce damages to human tissue. By providing the flexible portion 10 on the short side, on the basis of ensuring clamping strength between the stent and the tissue wall, overall anti-deformation performance of the stent may be effectively ensured, and the pull-apart resistance performance of the stent may be further ensured.

The foregoing descriptions are merely specific implementations of this application, but the protection scope of this application is not limited thereto. Any variation or replacement readily figured out by a person skilled in the art within the technical scope disclosed in this application shall fall within the protection scope of this application. Therefore, the protection scope of this application shall be subject to the protection scope of the claims.

What is claimed is:

1. A medical drainage stent, comprising:
a stent body, and a proximal portion and a distal portion that are located at two ends of the stent body for connect a first tissue wall and a second tissue wall in a juxtaposing manner, the stent body being fixed between the first tissue wall and the second tissue wall to provide a drainage channel,
wherein the proximal portion of the stent has N extension portions, a fixed end of the extension portion being fixedly connected with an edge of a proximal end of the stent body, and the other end of the extension portion being a free end in a free state; and
structures of the N extension portions at least partially protrude from the stent body in a radial direction, so that the proximal portion of the stent is fixed on the first tissue wall, wherein N is a positive integer.

2. The medical drainage stent according to claim 1, wherein the extension portion is an arc-shaped tube extending spirally along the edge of the proximal end of the stent body; the fixed end of the arc-shaped tube is fixed at the edge of the proximal end of the stent body; the other end is a free end in a free state; and a structure of the arc-shaped tube partially protrudes from the stent body in the radial direction, so that the proximal portion of the stent is fixed on the first tissue wall.

3. The medical drainage stent according to claim 1, wherein the extension portion extends outward along a radial direction of the stent body, the fixed end thereof is fixed at the edge of the proximal end of the stent body, and the other end is a free end in a free state.

4. The medical drainage stent according to claim 1, wherein all the extension portions are evenly arranged at the edge of the proximal end of the stent body, and an included angle between two adjacent extension portions is 360/N degrees, wherein N is a positive integer from 2 to 4.

5. The medical drainage stent according to claim 2, wherein all the extension portions are evenly arranged at the edge of the proximal end of the stent body, and an included angle between two adjacent extension portions is 360/N degrees, wherein N is a positive integer from 2 to 4.

6. The medical drainage stent according to claim 3, wherein all the extension portions are evenly arranged at the edge of the proximal end of the stent body, and an included angle between two adjacent extension portions is 360/N degrees, wherein N is a positive integer from 2 to 4.

7. The medical drainage stent according to claim 1, wherein the free end of the extension portion is provided with a fixing structure for fixedly connecting the free end to the first tissue wall, wherein the fixing structure and the free end are fixedly or not fixedly connected, and supporting force of the fixing structure is greater than that of the extension portion.

8. The medical drainage stent according to claim 2, wherein the free end of the extension portion is provided with a fixing structure for fixedly connecting the free end to the first tissue wall, wherein the fixing structure and the free end are fixedly or not fixedly connected, and supporting force of the fixing structure is greater than that of the extension portion.

9. The medical drainage stent according to claim 3, wherein the free end of the extension portion is provided with a fixing structure for fixedly connecting the free end to the first tissue wall, wherein the fixing structure and the free end are fixedly or not fixedly connected, and supporting force of the fixing structure is greater than that of the extension portion.

10. The medical drainage stent according to claim 1, further comprising at least one of the following structures:
a retrieval loop provided at the proximal portion and/or the distal portion of the stent; and
a developing member provided on the stent to display a position of the stent under rays.

11. The medical drainage stent according to claim 2, further comprising at least one of the following structures:
a retrieval loop provided at the proximal portion and/or the distal portion of the stent; and
a developing member provided on the stent to display a position of the stent under rays.

12. The medical drainage stent according to claim 3, wherein the stent comprising at least one of the following structures:
a retrieval loop provided at the proximal portion and/or the distal portion of the stent; and
a developing member provided on the stent to display a position of the stent under rays.

13. A medical drainage stent, comprising:
a stent body, and a proximal portion and a distal portion that are located at two ends of the stent body for connecting a first tissue wall and a second tissue wall in a juxtaposing manner, the stent body being fixed between the first tissue wall and the second tissue wall to provide a drainage channel,
wherein the stent body has a flexible portion whose supporting force is smaller than that of the proximal portion and the distal portion; and the flexible portion is able to form a recess with a reduced inner diameter after being pressed, so that structures of the proximal portion and the distal portion of the stent partially protrude from the stent body, and that the proximal portion of the stent is fixed on the first tissue wall, and the distal portion is fixed on the second tissue wall.

14. The medical drainage stent according to claim 13, wherein the stent body has more than two flexible portions that extend along an axial direction of the stent body, and the flexible portions are distributed at equal intervals in a circumferential direction of the stent body.

15. The medical drainage stent according to claim 13, wherein the flexible portion covers a part of a region of the stent body, and supporting force of the remaining region of the stent body is greater than that of the flexible portion.

16. The medical drainage stent according to claim 13, wherein the proximal portion and the distal portion of the stent have fixing structures protruding from the stent body.

17. The medical drainage stent according to claim 14, wherein the proximal portion and the distal portion of the stent have fixing structures protruding from the stent body.

18. The medical drainage stent according to claim 15, wherein the proximal portion and the distal portion of the stent have fixing structures protruding from the stent body.

19. The medical drainage stent according to claim 13, further comprising at least one of the following structures:
a retrieval loop provided at the proximal portion and/or the distal portion of the stent; and
a developing member provided on the stent to display a position of the stent under rays.

20. The medical drainage stent according to claim 14, wherein the stent further comprising at least one of the following structures:
a retrieval loop provided at the proximal portion and/or the distal portion of the stent; and
a developing member provided on the stent to display a position of the stent under rays.
